(19) United States
(12) Patent Application Publication
(10) Pub. No.: US 2021/0145842 A1
(43) Pub. Date: May 20, 2021

(54) NEW THERAPEUTIC USES

(71) Applicant: NOVARTIS AG, Basel (CH)

(72) Inventors: Susan Elizabeth MOODY, Cambridge, MA (US); Daniel Shao-Weng TAN, Singapore (SG)

(21) Appl. No.: 17/048,361

(22) PCT Filed: Apr. 17, 2019
(86) PCT No.: PCT/IB2019/053177

§ 371 Date: Oct. 16, 2020

Related U.S. Application Data
(60) Provisional application No. 62/678,651, filed on May 31, 2018.
(60) Provisional application No. 62/659,425, filed on Apr. 18, 2018.

(51) Int. Cl. A61K 31/55; A61P 35/04

(52) U.S. Cl. CPC A61K 31/55; A61P 35/04

(57) ABSTRACT

The present invention provides nazartinib, or a pharmaceutically acceptable salt thereof, preferably the mesylate salt thereof, for use in treating or preventing Central Nervous System (CNS) metastasis, brain metastasis, and/or leptomeningeal metastasis, particularly when the CNS or brain metastasis, or leptomeningeal metastasis is present in a patient with locally advanced or metastatic NSCLC.

The present invention provides nazartinib, or a pharmaceutically acceptable salt thereof, preferably the mesylate salt thereof, for use in treating or preventing a metastasis which is selected from Central Nervous System (CNS) metastasis, brain metastasis and leptomeningeal metastasis. In particular, the present invention provides these uses, wherein the metastasis is a result of a primary lesion such as non-small lung cancer (NSCLC) particularly, NSCLC which harbors an EGFR mutation (exon 19 deletion or exon 21 (L858R) substitution). This invention may be particularly useful in the first-line treatment of patients suffering from NSCLC, including locally advanced or metastatic NSCLC, which harbors an EGFR mutation (exon 19 deletion or exon 21 (L858R) substitution).

### BACKGROUND

Lung cancer is the most common and deadly cancer worldwide, with non-small cell lung cancer (NSCLC) accounting for approximately 85% of lung cancer cases. In Western countries, 10-15% non-small cell lung cancer (NSCLC) patients express epidermal growth factor receptor (EGFR) mutations in their tumors and Asian countries have reported rates as high as 30-40%. The predominant oncogenic EGFR mutations (L858R and ex19del) account for about 85% of EGFR-mutant NSCLC.

EGFR-mutant patients are given an EFGR inhibitor as first line therapy. However, most patients develop acquired resistance, generally within 10 to 14 months. In up to 50% of NSCLC patients harboring a primary EGFR mutation treated with first generation reversible EGFR tyrosine kinase inhibitors (TKIs), also referred to as first-generation TKIs, such as erlotinib, gefitinib and icotinib, a secondary “gatekeeper” T790M mutation develops.

Second-generation EGFR TKIs (such as afatinib and dacomitinib) have been developed to try to overcome this mechanism of resistance. These are irreversible agents that covalently bind to cysteine 797 at the EGFR ATP site. Second generation EGFR TKIs are potent on both activating [L858R, ex19del] and acquired T790M mutations in pre-clinical models. Their clinical efficacy has however proven to be limited, possibly due to severe adverse effects caused by concomitant wild-type (WT) EGFR inhibition. Resistance to second-generation inhibitors also soon develops, with virtually all patients receiving first- and second-generation TKIs becoming resistant to any of the first or second generation TKI after approximately 9-13 months.

This has led to the development of third-generation EGFR TKIs, e.g. nazartinib (EGF816), rociletinib, ASP8273 and osimertinib (Tagrisso®). Third-generation EGFR TKIs are WT EGFR sparing and also have relative equal inhibitory potency for activating EGFR mutations [L858R, ex19del] and acquired T790M mutations. Osimertinib has recently been approved in the United States for the treatment of patients with advanced EGFR T790M+ NSCLC whose disease has progressed on or after an EGFR TKI therapy. Osimertinib demonstrates some wild-type EGFR inhibition.

Patients harboring EGFR-mutant tumors often progress during TKI treatment due to growth of secondary brain metastases (Porta et al. Eur Respir J 2011;37:624-31). In addition, CNS metastases and brain metastases are common in patients with advanced NSCLC. More than 30% of patients with NSCLC experience disease progression during treatment with established EGFR-TKIs due to growth of synchronous or metachronous brain metastases (Ballard et al. 2016, Clin Cancer Res. 2016, 22(20):5130-5140). In addition, there is a cumulative increase in brain metastases incidence in patients with EGFR-mutant NSCLC over time (Rangachari et al. Lung Cancer 2015;88:108-11). Treatment for CNS metastases and brain metastases from NSCLC includes surgical resection, stereotactic radiosurgery and whole-brain radiotherapy (WRBT). Although many patients die of systemic progression, rather than brain lesion progression, quality of life is significantly worsened, both directly and as a result of whole brain radiotherapy (WBRT), which degrades cognitive function (Li at al. Int J Radiat Oncol Biol Phys 2008;71:64-70). Therefore, there is a clinical need for novel EGFR-tyrosine kinase inhibitors (EGFR-TKI) with improved efficacy against malignancies in the central nervous system and against brain lesions, whilst providing efficacious and durable responses in patients with EGFR-mutant NSCLC, including patients with baseline brain metastases. There is also a need to provide a good quality of life for patients suffering from NSCLC, specially for such patients who are at risk of developing brain metastases.

### SUMMARY OF THE INVENTION

Nazartinib is a compound of formula I below

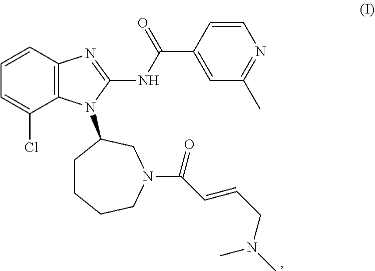 [SMILES: Cc1cc(C(=O)Nc2nc3cccc(Cl)c3n2[C@@H]2CCCCN(C(=O)/C=C/CN(C)C)C2)ccn1]

which has the chemical name of (R,E)-N-(7-chloro-1-(1-(4-(dimethylamino)but-2-enoyl)azepan-3-yl)-1H-benzo [d]imidazol-2-yl)-2-methylisonicotinamide.

It has been shown that nazartinib showed clinical efficacy with good brain penetration in the treatment of patients with NSCLC, despite the distribution of nazartinib to the brain being found minimal in a preclinical model. Patients treated with nazartinib may hence achieve stable disease state and may be slower to progress than when treated with standard of care treatment, such as erlotinib or gefitinib. Thus, nazartinib may have clinical benefit in patients suffering from NSCLC. The present invention provides an improved therapy for patients with NSCLC, e.g., locally advanced or metastatic NSCLC, who may or may not be treatment naïve and whose tumors harbor an EGFR activating mutation (L858R or ex19del). The present therapy may also be useful in the prevention of Central Nervous System (CNS) metastasis, brain metastasis and leptomeningeal metastasis in patients suffering from NSCLC.

The present invention thus provides the following:
a method of treating a patient having NSCLC, comprising selectively administering a therapeutically effective amount of nazartinib, or a pharmaceutically acceptable salt thereof, to a patient having previously been determined to have an exon 19 deletion or exon 21 (L858R) substitution EGFR mutation;
a method of treating a patient having NSCLC, comprising: (a) determining or having determined that the patient has an exon 19 deletion or exon 21 (L858R) substitution EGFR mutation; and (b) administering a therapeutically effective amount of nazartinib, or a pharmaceutically acceptable salt thereof, to said patient;
a method of treating a patient having NSCLC, comprising selecting a patient for treatment based on the patient having been previously determined to have an exon 19 deletion or exon 21 (L858R) substitution EGFR mutation, and administering a therapeutically effective amount of nazartinib, or a pharmaceutically acceptable salt thereof, to said patient.

The patient may be a patient with NSCLC whose cancer has progressed to a metastasis which is selected from central nervous system (CNS) metastasis, brain metastasis and leptomeningeal metastasis. The metastasis is preferably brain metastasis.

The present invention thus provides nazartinib, or a pharmaceutically acceptable salt thereof, preferably the mesylate salt thereof, or a pharmaceutical composition comprising such a compound, for use in treating or preventing CNS metastasis, brain metastasis and/or leptomeningeal metastasis. In one embodiment, the CNS, brain metastasis, or leptomeningeal metastasis is present in a patient suffering from locally advanced or metastatic NSCLC. In a preferred embodiment, the patient has metastatic NSCLC, optionally where the NSCLC harbors an EGFR mutation (exon 19 deletion or exon 21 (L858R) substitution mutation). Such a patient may also have progressed to develop CNS metastasis, brain metastasis, and/or leptomeningeal metastasis.

The present invention provides a compound which is nazartinib, or a pharmaceutically acceptable salt thereof, for use in treating or preventing a metastasis in a patient, wherein the metastasis is selected from central nervous system (CNS) metastasis, brain metastasis and leptomeningeal metastasis, optionally wherein the metastasis is a result of a primary lesion such as non-small lung cancer (NSCLC) particularly, wherein the patient has been predetermined to have NSCLC which harbors an EGFR mutation (exon 19 deletion or exon 21 (L858R) substitution).

The present invention provides nazartinib, or a pharmaceutically acceptable salt thereof, for use in treating or preventing NSCLC, wherein the patient is suffering from a metastasis wherein the metastasis is selected from central nervous system (CNS) metastasis, brain metastasis and leptomeningeal metastasis, wherein the patient has been predetermined to have NSCLC which harbors an EGFR mutation (exon 19 deletion or exon 21 (L858R) substitution).

The present invention provides nazartinib, or a pharmaceutically acceptable salt thereof, preferably the mesylate salt thereof, for use in treating NSCLC, optionally locally advanced or metastatic NSCLC, with EGFR activating mutations (e.g. L858R or ex19del) in a patient, for example, wherein:
(a) the Progression Free Survival (PFS) of the patient is improved, e.g., in relation to the PFS obtained following treatment with erlotinib or gefitinib; or
(b) the overall survival (OS) of the patient is improved, e.g. in relation to the OS obtained following treatment with erlotinib, erlotinib or gefitinib; or
(c) the overall response rate (ORR) of the patient is improved in relation to the ORR obtained following treatment with erlotinib, erlotinib or gefitinib; or
(d) the time to progression (TPS) in CNS or in the brain is increased; or
(e) the CNS ORR or brain ORR is increased; or
(f) the CNS duration of response (DoR) or brain DoR is increased;
   including any combinations of the features from (a) to (f)

As disclosed herein, nazartinib may provide a therapeutic benefit selected from the group consisting of effects (a) to (f) above, and any combinations thereof.

In one preferred embodiment, nazartinib is used as monotherapy for the treatment of EGFR-mutant NSCLC. In another preferred embodiment, nazartinib is used as part of a combination therapy for the treatment of EGFR-mutant NSCLC, e.g., for the treatment-naive patients with advanced EFGR-mutant NSCLC, including patients with brain metastases.

In particular, nazartinib as single agent may be useful in
providing long term benefit to patients suffering from NSCLC, e.g., patients with treatment-naïve locally advanced or metastatic NSCLC tumors harboring EGFR activating mutations;
providing increased time to progression in the CNS or the brain, ORR in the CNS or the brain, and DoR in the CNS or in the brain as determined by central neuro-radiologist BIRC according to modified RECIST 1.1 for patients with CNS disease or brain lesions at baseline;
improved patient reported outcomes (PRO), including patients' disease-related symptoms and Health Related Quality of Life (HRQoL), specially compared to erlotinib or gefitinib improved safety and tolerability profile of single agent nazartinib , e.g. compared to erlotinib or gefitinib;
   or any combination of the above benefits.

In another embodiment, nazartinib is used in combination with another antineoplastic medicament.

An object of the present invention is therefore to provide a therapy to improve the treatment of a cancer, particularly non-small cell lung cancer (NSCLC), more particularly an EGFR-mutant NSCLC. In particular, the aim of the present invention is to provide a safe and tolerable treatment which prevents or delays the emergence or progression of CNS metastasis, particularly brain metastasis. Nazartinib, as described herein, was found to be tolerable with an acceptable safety profile in patients with EGFR-mutant NSCLC, increased time to progression in the CNS or in the brain ORR in the CNS or in the brain, and DoR in the CNS or in the brain as determined by central neuro-radiologist BIRC according to modified RECIST 1.1 for patients with CNS disease at baseline.

### DETAILED DESCRIPTION OF THE FIGURE

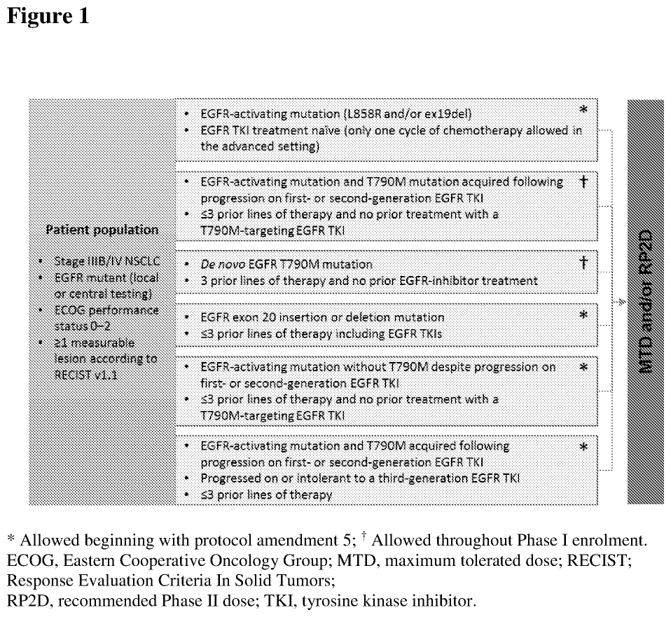

FIG. 1: Phase I study design

### DETAILED DESCRIPTION OF THE INVENTION

In one aspect, the present invention relates to a pharmaceutical composition comprising nazartinib, or a pharmaceutically acceptable salt thereof, for use in treating or preventing a metastasis which is selected from CNS metastasis, brain metastasis and leptomeningeal metastasis.

In one embodiment, nazartinib is in its mesylate salt form.

In a preferred embodiment, the CNS or leptomeningeal metastasis is present in a patient with locally advanced or metastatic NSCLC.

In one embodiment, the tumor harbors an EGFR-activating mutation, preferably a L858R mutation and/or a ex19del mutation.

In one embodiment, the patient is an EGFR T790M mutation-positive NSCLC patient who has progressed to develop CNS metastasis and/or leptomeningeal metastasis.

In another aspect, the present invention provides nazartinib, or a pharmaceutically acceptable salt thereof, preferably the mesylate salt thereof, for use in treating NSCLC, optionally locally advanced or metastatic NSCLC, with EGFR activating mutations (e.g. L858R or ex19del) in a patient, wherein:
(a) the Progression Free Survival (PFS) of the patient is improved, e.g. in relation to the PFS obtained following treatment with erlotinib or gefitinib, or comparable with PFS obtained following treatment with erlotinib or gefitinib. PFS may be measured using a central blinded independent review committee (BIRC) according to Response Evaluation Criteria in Solid Tumors (RECIST 1.1)
(b) the overall survival (OS) of the patient is improved, e.g. in relation to the OS obtained following treatment with erlotinib or gefitinib, or comparable with PFS obtained following treatment with erlotinib or gefitinib, or
(c) the overall response rate (ORR, measured by central BRIC) of the patient is improved in relation to the ORR obtained following treatment with erlotinib or gefitinib, or comparable with the ORR obtained following treatment with erlotinib or gefitinib, or
(d) the time to progression in Central Nervous System (CNS) is increased (e.g. in comparison with the standard of care, or following treatment with erlotinib, gefitinib or osimertinib), or
(e) the CNS ORR is increased (CNS ORR may be measured per central neuro-radiologist BIRC), e.g. in patients with brain metastases who have measurable disease in the brain at baseline review per modified RECIST 1.1, or
(f) the CNS Duration of response (DoR) is increased (e.g., per central neuro-radiologist BIRC; e.g. CNS DoR in patients with brain metastases who have measurable disease in the brain at baseline per modified RECIST 1.1).

As disclosed herein, nazartinib as single agent may provide a therapeutic benefit selected from the group consisting of effects (a) to (f) above, and any combination thereof. In addition, nazartinib may offer an improved treatment option with fewer side-effects as compared to treatment with gefitinib, erlotinib or osimertinib treatment.

In one embodiment, nazartinib is used as monotherapy. In another embodiment, nazartinib is used in combination with another antineoplastic medicament.

Nazartinib

Nazartinib and pharmaceutically acceptable salts thereof are described in WO2013/184757.

Nazartinib is also known by the code name “EGF816”. As used herein, the terms “nazartinib” or “EGF816” refer to a compound of formula (I), or a pharmaceutically acceptable salt thereof, unless otherwise indicated herein or clearly contradicted by context.

Nazartinib is a targeted covalent irreversible inhibitor of Epidermal Growth Factor Receptor (EGFR) that selectively inhibits activating and acquired resistance mutants (L858R, ex19del and T790M), while sparing wild type (WT) EGFR (Jia et al, Cancer Res October 1, 2014 74; 1734). Nazartinib has shown significant efficacy in EGFR mutant (L858R, ex19del and T790M) cancer models (in vitro and in vivo) with no indication of WT EGFR inhibition at clinically relevant efficacious concentrations. Dose-dependent anti-tumor efficacy was observed in several xenograft models and nazartinib was well tolerated with no body weight loss observed at efficacious doses.

Nazartinib was found to show durable antitumor activity in a clinical study with patients suffering from advanced non-small cell lung cancer (NSCLC) harboring T790M (Tan et al, Journal of Clinical Oncology 34, no. 15_suppl (May 2016)).

Pharmaceutical compositions comprising nazartinib, or a pharmaceutically acceptable salt thereof, are described in WO2013/184757 which is incorporated by reference herein in its entirety. Nazartinib, or its pharmaceutically acceptable salt, may be administered as an oral pharmaceutical composition in the form of a capsule formulation or a tablet. Pharmaceutically acceptable salts of nazartinib include the mesylate salt and the hydrochloride salt thereof. Preferably the pharmaceutically acceptable salt is the mesylate salt.

It is envisaged that nazartinib may be particularly useful for patients who are treatment naïve patients, i.e. patients who have not received any prior therapy for NSCLC, e.g. advanced NSCLC. “Treatment naïve” patients include patients who are treatment naive from any systemic antineoplastic therapy in the advanced setting including chemotherapy, biologic therapy, immunotherapy or any investigational therapy. This term also describes patients who received previous neo-adjuvant or adjuvant systemic therapy with a relapse which has occurred more than 12 months from the end of the neo-adjuvant or adjuvant systemic therapy, whichever is the later. It is also envisaged that these patients include third-generation EGFR TKI-naïve patients.

Thus, the present invention also provides a compound for use and a composition as described herein for use in the first-line treatment of non-small cell lung cancer, including EGFR-mutant NSCLC (e.g., exon 19 deletion or L858R mutation NSCLC).

The present invention also provides the use of nazartinib, or a pharmaceutically acceptable salt for the manufacture of a medicament for the first-line treatment of non-small lung cancer, including EGFR-mutant (e.g. exon 19 deletion or L858R mutation NSCLC).

Patients likely to benefit from the uses, pharmaceutical compositions and the therapeutic regimens provided herein also include pre-treated patients, e.g., patients who have received prior treatment with a first-generation EGFR TKI and/or a second generation EGFR TKI.

Patients likely to benefit from the present invention also include patients who have been prescreened for HBV and HCV infection and are found to be free from such infection or who are prescribed concurrent treatment with an antiviral treatment. For example, patients who are either HBsAg positive or HBV-DNA positive may take antiviral therapy (e.g. entecavir or tenofovir) or at least 1 week prior to first dose of nazartinib treatment and continue on antiviral therapy for at least 4 weeks after the last dose of nazartinib.

Tumor burden (also called “tumor load”) refers to the number of cancer cells, the size of a tumor, or the amount of cancer in the body. A subject suffering from cancer is defined as having progressed on, or no longer responding to therapy with one or more agents, or being intolerant to with one or more agents when the cancer he or she is suffering from, has progressed i.e., the tumor burden has increased. Progression of cancer such as NSCLC or tumors may be indicated by detection of new tumors or detection of metastasis or cessation of tumor shrinkage. The progression of cancer and the assessment of tumor burden increase or decrease may be monitored by methods well known to those in the art. For example, the progression may be monitored by way of visual inspection of the cancer, such as, by means of X-ray, CT scan or MRI or by tumor biomarker detection. An increased growth of the cancer may indicate progression of the cancer. Assessment of tumor burden may be determined by the percent change from baseline in the sum of diameters of target lesions. Tumor burden assessment, whereby a decrease or increase in tumor burden is determined, will normally be carried out at various intervals, e.g., in successive assessments carried out at least 1, 2, 3 month(s), preferably one month apart.

Tumor evaluations and assessment of tumor burden can be made based on RECIST criteria (Therasse et al 2000), New Guidelines to Evaluate the Response to Treatment in Solid Tumors, Journal of National Cancer Institute, Vol. 92; 205-16 and revised RECIST guidelines (version 1.1) (Eisenhauer et al 2009) European Journal of Cancer; 45:228-247.

The pharmaceutical composition disclosed herein is particularly useful for the treatment of a of non-small cell lung cancer (NSCLC). The most common types of NSCLC are squamous cell carcinoma, large cell carcinoma, and lung adenocarcinoma. Less common types of NSCLC include pleomorphic, carcinoid tumor, salivary gland sarcoma, and unclassified sarcoma. The NSCLC, and in particular lung adenocarcinoma, may be characterized by aberrant activation of EGFR, in particular amplification of EGFR, or somatic mutation of EGFR.

The lung cancer to be treated thus includes EGFR mutant NSCLC. It is envisaged that nazartinib or a pharmaceutical composition comprising nazartinib or a pharmaceutically acceptable salt thereof, will be useful in treating advanced EGFR mutant NSCLC. Advanced NSCLC refers to patients with either locally advanced or metastatic NSCLC. Locally advanced NSCLC is defined as stage IIIB NSCLC not amenable to definitive multi-modality therapy including surgery. Metastatic NSCLC refers to stage IV NSCLC.

EGFR mutation status may be determined by tests available in the art, e.g. QIAGEN therascreen® EGFR test or other FDA approved tests. The therascreen EGFR RGQ PCR Kit is an FDA-approved, qualitative real-time PCR assay for the detection of specific mutations in the EGFR oncogene. Evidence of EGFR mutation can be obtained from existing local data and testing of tumor samples. EGFR mutation status may be determined from any available tumor tissue.

Nazartinib may be particularly useful for treating NSCLC which harbors an EGFR L858R mutation, an EGFR exon 19 deletion or both. The NSCLC to be treated may also harbor a further EGFR T790M mutation which may be a de novo mutation or an acquired mutation.

In one embodiment, the EGFR T790M mutation is a de novo mutation. The term “de novo mutation” is defined herein to refer to an alteration in a gene that is detectable or detected in a human, before the onset of any treatment with an EGFR inhibitor. A de novo mutation is a mutation which normally has occurred due to an error in the copying of genetic material or an error in cell division, e.g., de novo mutation may result from a mutation in a germ cell (egg or sperm) of one of the parents or in the fertilized egg itself, or from a mutation occurring in a somatic cell.

A “de novo” T790M mutation is defined as the presence of EGFR T790M mutation in NSCLC patients who have NOT been previously treated with any therapy known to inhibit EGFR.

In another aspect, the present invention relates to the pharmaceutical composition comprising nazartinib and at least one pharmaceutically acceptable carrier.

As used herein, the term “pharmaceutically acceptable carrier” includes generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences). Except insofar as any conventional carrier is incompatible with nazartinib its use in the pharmaceutical compositions or medicaments is contemplated.

Examples of pharmaceutical compositions comprising nazartinib, or a pharmaceutically acceptable salt thereof, are described in WO2013/184757.

Dosages

The dosages or doses quoted herein, unless explicitly mentioned otherwise, refer to the amount present, in the drug product, of nazartinib, calculated as the free base.

When nazartinib is administered as monotherapy or as part of a combination therapy, the dose of nazartinib may be selected from a range of about 50 to about 200 mg, more preferably from a range of about 50 to about 150 mg. nazartinib may be administered at a dosage of about 25, about 50, about 75, about 100, or about 150 mg once daily. Thus, nazartinib may be administered at a dosage of about 50, about 75, about 100 or about 150 mg once daily; more preferably, about 50, about 75 or about 100 mg once daily. The about 50, about 75 or about 100 mg doses may be better tolerated without loss of efficacy. In a preferred embodiment, nazartinib may be administered at a dosage of about 100 mg or about 150 mg once daily. Hepatitis reactivation is not expected to occur at a dose of about 150 mg or less daily.

The term “effective amount” or “therapeutically effective amount” of a or the therapeutic agent is defined herein to refer to an amount sufficient to provide an observable improvement over the baseline clinically observable signs and symptoms of the cancer treated with the therapeutic agent.

Determination of the attainment of stable disease response may be determined by using Response Evaluation Criteria In Solid Tumors (RECIST 1.1) or WHO criteria. A Stable Disease (SD) response may be defined as a response where the target lesions show neither sufficient shrinkage to qualify for Partial Response (PR) nor sufficient increase to qualify for Progressive Disease (PD), taking as reference the smallest sum Longest Diameter (LD) of the target lesions since the treatment started. Other Response Criteria may be defined as follows.
Complete Response (CR): Disappearance of all target lesions
Partial Response (PR): At least a 30% decrease in the sum of the LD of target lesions, taking as reference the baseline sum LD.
Progressive Disease (PD): At least a 20% increase in the sum of the LD of target lesions, taking as reference the smallest sum LD recorded since the treatment started or the appearance of one or more new lesions.

The treatment period during which the EGFR inhibitor as monotherapy or combination therapy is administered may thus be readily measured by a skilled person in the art. The treatment period may consist of one, two, three, four, five, six or more 14-day, 21-day, 28-day or 35-day cycles, preferably two or three cycles. Cycles are preferably 21-day or 28-day cycles.

Definitions

The term “pharmaceutically acceptable salt” refers to a salt that retains the biological effectiveness and properties of the compound and which typically is not biologically or otherwise undesirable. The compound may be capable of forming acid addition salts by virtue of the presence of an amino group.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “about” refers to a statistically acceptable variation in a given value, and typically is +/−5% or 10% , On the other hand, when a numerical value is quoted without being accompanied by the term “about”, it will be understood that this numerical value will include a variation of that value which is statistically acceptable in the art.

The terms “treat”, “treating” and “treatment” are defined herein to refer to a treatment relieving, reducing or alleviating at least one symptom in a subject or affecting a delay of progression of a disease. For example, treatment can be the diminishment of one or several symptoms of a disease or complete eradication of a disease, such as cancer. Within the meaning of the present invention, the term “treat”, “treatment” and “improved treatment” also denote one or more of the following: iimproving PFS, improving OS, ORR, increasing the time to progression in CNS or the brain, a increasing ORR and/or DoR in CNS or the brain, specially as compared to treatment with gefitinib and/or erlotinib.

The term “subject” or “patient” as used herein refers to a human suffering from a cancer, preferably lung cancer, e.g., NSCLC, in particular, EGFR mutant NSCLC.

As used herein, “select”, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology.

The term “determining” refers to the act of carrying out a test, procedure, experiment, assay, analysis, etc. to define the presence (or absence) of a given marker, e.g., a biomarker or genetic mutation, e.g., a T790M, exon 19 deletion, or exon 21 (L858R) substitution EGFR mutation, in a biological sample (e.g., a sample from a tumor metastasis) from a patient.

The phrase “having determined” refers to the act of requesting a third party (e.g., lab, hospital, nurse, physician) to carry out or provide results from a test, procedure, experiment, assay, analysis, etc. that defines the presence (or absence) of a given marker, e.g., a biomarker or genetic mutation, e.g., a T790M, exon 19 deletion, or exon 21 (L858R) substitution EGFR mutation in a biological sample (e.g., a sample from a tumor metastasis), from a patient.

The phrase “having been previously determined” refers to the status (e.g., genetic status, patient characteristics, biomarker status, etc.) of a given patient that has already been identified in the past by some party (third party or otherwise).

The term “monotherapy” as used herein refers to the use of nazartinib (as the free base or as its pharmaceutically acceptable salt) as a single drug to treat the disease or condition. Thus the term “monotherapy” as used herein does not include the use of nazartinib with another therapeutic drug.

The terms “combination therapy” or “combination” and such like, do not imply that the therapy or the therapeutic agents must be physically mixed or administered at the same time and/or formulated for delivery together, although these methods of delivery are within the scope described herein. A therapeutic agent in these combinations can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized as single-agent therapeutics.

PFS is defined as the time from the date of randomization to the date of the first documented progression or death due to any cause, whichever occurs first. PFS is usually assessed via local review according to RECIST 1.1.

PFS per local review may be analyzed using a stratified Cox model, with the same analysis conventions as the primary efficacy analysis. The PFS distribution is typically estimated using the Kaplan-Meier method, and the Kaplan-Meier curves, medians and 95% confidence intervals of the medians may be presented for each treatment group. The hazard ratio for PFS may be calculated, along with its 95% confidence interval, using a stratified Cox model. Concordance analysis between local and central BICR review of PFS may be provided by treatment group.

The PFS obtained following treatment with erlotinib, gefitinib or osimertinib may be from 9 to 10 months, e.g. 9.7, 9.5 months and 10.2 months, respectively.

ORR is defined as the proportion of patients with BOR (Best Overall Response) of CR (Complete Response) or PR (Partial Response), as per central BIRC review and according to RECIST 1.1. ORR are calculated based on the FAS and according to the ITT (Intention-to-treat) principle.

BOR for each patient is determined from the sequence of overall (lesion) responses according to the following rules:
CR=at least two determinations of CR at least 4 weeks apart before progression.
PR=at least two determinations of PR or better at least 4 weeks apart before progression (and not qualifying for a CR).
SD=at least one SD assessment (or better) >6 weeks after randomization (and not qualifying for CR or PR).
PD=progression ≤12 weeks after randomization (and not qualifying for CR, PR or SD). ORR and its 95% confidence interval imay be presented by treatment group.

As supportive analysis, the above analysis may be carried out using investigators assessments. Concordance analysis between local and central BICR review of BOR may be provided by treatment group.

CNS ORR or brain ORR only applies to patients with measurable disease in the brain at baseline by central neuro-radiologist BIRC per modified RECIST 1.1. CNS ORR or brain ORR is defined as the proportion of patients with best overall response of CR or PR in the brain.

DCR (Disease control rate) is defined as the proportion of patients with confirmed best overall response (BOR) of CR, PR, or SD. CR, PR and SD are defined as per central BIRC review according to RECIST 1.1. DCR may be calculated based on the FAS and according to the ITT principle. DCR and its 95% confidence interval may be presented by treatment group.

As supportive analysis, the above analysis may be carried out using investigators assessments.

TTR (Time to response) is defined as the time from the date of randomization to the first documented response (CR or PR, which must be subsequently confirmed). CR and PR are based on tumor response data as per central BIRC review according to RECIST 1.1. All patients in the FAS (Full Analysis Set) are included in TTR calculations. Patients without a confirmed CR or PR will be censored at the study-maximum follow-up time (i.e., LPLV-FPFV) for patients with a PFS event (i.e., disease progression or death due to any cause), or at the date of the last adequate tumor assessment for patients without a PFS event. TTR may be listed and summarized by treatment group. The TTR distribution is estimated using the Kaplan-Meier method. The medians and 95% confidence intervals of the medians may be presented for each treatment group.

Time to progression (TTP) is the time from date of randomization/start of treatment to the date of event defined as the first documented progression or death due to underlying cancer. If a patient has not had an event, time to progression is censored at the date of last adequate tumor assessment. Time to progression in the CNS or the brain is defined as the time from date of randomization to the date of first documented progression of brain metastases as assessed by central neuro-radiologist BIRC per modified RECIST 1.1 for FAS patients with at least one non-measurable and/or measurable disease in the brain at baseline.

Analysis of time to progression in the CNS or in the brain, CNS ORR, brain ORR, brain DoR and CNS DoR is carried out in a same manner as described above for PFS, ORR and DoR respectively. The stratification may be based on the randomization stratification factors, i.e. race (Asian vs non-Asian), and EGFR activating mutation type (L858R vs ex19del). DoR (Duration of response) only applies to patients whose best overall response is CR or PR according to RECIST 1.1 based on tumor response data per central BIRC review. The start date is the date of first documented response (CR or PR), and the end date is defined as the date of the first documented progression or death due to underlying cancer, whichever occurs first. Patients continuing without progression or death due to underlying cancer are censored at the date of their last adequate tumor assessment. DoR may be listed and summarized by treatment group for all patients in the FAS with confirmed BOR of CR or PR.

The DoR distribution may be estimated using the Kaplan-Meier method. The medians and 95% confidence intervals of the medians may be presented for each treatment group.

CNS DoR or brain DoR only applies to patients with measurable disease in the brain at baseline and whose best overall response in the CNS or in the brain is CR or PR according to modified RECIST 1.1 based on tumor response data per central neuro-radiologist BIRC review. The start date is the date of first documented response (CR or PR) in the brain, and the end date is defined as the date of the first documented progression in the brain. Patients continuing without progression in the brain are censored at the date of their last adequate tumor assessment.

Clinical benefit rate (CBR) is the proportion of patients with a best overall response of CR or PR, or an overall lesion response of SD or Non-CR/Non-PD which lasts for a minimum time duration (with a default of at least 24 weeks in breast cancer studies). This endpoint measures signs of activity taking into account duration of disease stabilization.

PFS2: A recent EMA guidance (EMA 2012) recommends a substitute end point intermediate to PFS and OS called “PFS2”, a surrogate for OS when OS cannot be measured reliably, which assesses the impact of the experimental therapy on next-line treatment. The main purpose of this endpoint is to assess long term maintenance strategies, particularly of resensitizing agents and where it is necessary to examine the overall “field of influence”.

PFS2, which could be termed “PFS deferred”, “PFS delayed”, “tandem PFS”, or “PFS version 2.0”, is the time from date of randomization/start of treatment to the date of event defined as the first documented progression on next-line treatment or death from any cause. The censoring rules for this endpoint incorporate the same principles as those considered for PFS.

Determination of Target Lesion Response

Complete Response (CR) is defined as disappearance of all non-nodal target lesions. In addition, any pathological lymph nodes assigned as target lesions must have a reduction in short axis to <10 mm. SOD for CR may not be zero when nodal lesions are part of target lesions.

Partial Response (PR) is defined as at least a 30% decrease in the sum of diameter of all target lesions, taking as reference the baseline sum of diameters.

Progressive Disease (PD) is defined as at least a 20% increase in the sum of diameter of all measured target lesions, taking as reference the smallest sum of diameter of all target lesions recorded at or after baseline. In addition to the relative increase of 20%, the sum must also demonstrate an absolute increase of at least 5 mm. Following an initial CR, a PD cannot be assigned if all non-nodal target lesions are still not present and all nodal lesions are <10 mm in size. In this case, the target lesion response is CR.

Stable Disease (SD) is defined as neither sufficient shrinkage to qualify for PR or CR nor an increase in lesions which would qualify for PD.

Unknown (UNK) is defined as progression has not been documented and one or more target lesions have not been assessed or have been assessed using a different method than baseline. In exceptional circumstances an UNK response due to change in method could be over-ruled by the investigator or central reviewer using expert judgment based on the available information.

Determination of Non-Target Lesion Response

CR is defined as D=disappearance of all non-target lesions. In addition, all lymph nodes assigned a non-target lesions must be non-pathological in size (<10 mm short axis).

PD is defined as unequivocal progression of existing non-target lesions. The assignment of PD solely based on change in non-target lesions in light of target lesion response of CR, PR or SD should be exceptional. In such circumstances, the opinion of the investigator or central reviewer does prevail.

Unknown (UNK) is defined as progression has not been documented and one or more non-target lesions have not been assessed or have been assessed using a different method than baseline. It is recommended that the investigator and/or central reviewer should use expert judgment to assign a Non-UNK response wherever possible (see notes section for more details).

Disease progression in the brain may be defined as a new brain lesion, worsening of a baseline brain non-target lesion, or ≥20% increase in sum of longest diameters of baseline brain target lesions.

The following Examples illustrate the invention described above, but are not, however, intended to limit the scope of the invention in any way. Other test models known to the person skilled in the pertinent art can also determine the beneficial effects of the claimed invention.

### EXAMPLES

### Example 1

### Phase I, Multicenter, Open-Label Study of Nazartinib (EGF816) in Adult Patients with EGFR-Mutant NSCLC

Acquired resistance to first-generation epidermal growth factor receptor (EGFR) tyrosine kinase inhibitors (TKIs) through T790M “gatekeeper” mutation occurs in 50-60% of treated patients with non-small cell lung cancer (NSCLC). Nazartinib (EGF816), a third-generation EGFR TKI selective for activating and T790M mutations while sparing wild-type EGFR, was evaluated in patients with advanced NSCLC harboring an EGFR mutation.

Patients had stage IIIB/IV EGFR-mutant NSCLC, ≥1 measurable lesion, and Eastern Cooperative Oncology Group performance status ≤2. Patients were screened for eligibility across six subgroups according to EGFR mutation status and prior therapy, and treated with nazartinib 75-350 mg (capsule or tablet formulation) orally, once daily (QD), on a continuous 28-day dosing schedule. The primary objective of the Phase I part was to determine the maximum tolerated dose and/or recommended Phase II dose (RP2D).

By August 2017, 180 patients were treated with nazartinib across seven dose levels. Nazartinib was found to be tolerable with an acceptable safety profile in patients with EGFR-mutant NSCLC. The best overall response rate across doses, based on 162 evaluable patients with EGFR T790M-positive tumors who were naïve to third-generation EGFR TKIs, was 51% (95% CI: 43-59) with a 11.0-month median duration of response. The median progression-free survival (PFS) was 9.1 months (95% CI: 7.3-11.1). Clinical benefit was experienced in most patients treated with nazartinib, with an overall disease control rate of 89% in patients with EGFR T790M-positive tumors who were naïve to third-generation EGFR TKIs. Out of 162 such patients, 45 patients (28%) had detectable brain metastases recorded as non-target lesions at baseline, and seven (16%) of these patients displayed resolution of brain lesions while on study treatment. These data demonstrate clinically relevant anti-tumor activity of nazartinib, including in the brain, in patients with EGFR-mutant NSCLC.

Therefore, given the limited treatment options available for patients with advanced EGFR-mutant NSCLC, nazartinib may provide an effective treatment option in this setting.

Methods

Study Design

This was a Phase I/II, multicenter, open-label study of nazartinib in patients with locally advanced/metastatic EGFR-mutant NSCLC.

Nazartinib was administered orally, once daily (QD), on a continuous, 28-day cycle dosing schedule. The starting dose for the Phase I part was 75 mg in capsule formulation. Nazartinib in tablet formulation was also introduced during Phase I. Patients were treated across the following QD dose groups: 75 mg, 100 mg, 150 mg, 200 mg, 225 mg, 300 mg, and 350 mg. Initial cohorts consisted of 1-6 patients.

Statistical Analyses

Patients treated with capsule or with tablet at the same dose level were pooled together into a single treatment group for safety and efficacy analyses. The Full Analysis Set (FAS) consists of all patients who received ≥1 dose of the study drug. Efficacy analyses were performed on all patients in the FAS who had baseline and post-baseline tumor assessment data or who had discontinued prior to post-baseline tumor assessment, and excluded patients with tumors that were wild-type at EGFR T790 or who had received prior third-generation EGFR TKIs, unless otherwise noted.

Duration of response (DoR) and PFS were described using the Kaplan-Meier method. Data from 180 patients enrolled in the Phase I dose-escalation part are reported in this study.

Results

At the data cut-off date of Aug. 31, 2017, 180 patients had been enrolled and treated with nazartinib across seven QD dose levels in either tablet or capsule formulation (75 mg [n=17], 100 mg [n=38], 150 mg [n=73], 200 mg [n=8], 225 mg [n=28], 300 mg [n=5], and 350 mg [n=11]).

Recommended Dose

The 150 mg QD (once daily) dose level was well tolerated and demonstrated good antitumor efficacy, with an ORR of 46%. Based on these observations, 150 mg QD was selected as the recommended phase II dose (RP2D).

Other doses of nazartinib, as described above, may also be useful in the present invention.

Discussion

In this study, nazartinib demonstrated clinical activity in patients with EGFR T790M-mutated NSCLC, with most patients across all dose levels deriving clinical benefit. Nearly half of all evaluable patients achieved an objective response and the DCR was 87%. The median duration of response was 11.0 months, comparable to the 9.7-month median duration of response reported in patients with EGFR T790M⁺ NSCLC treated with osimertinib in the Phase III AURA3 study (Mok et al. N Engl J Med 2017; 376: 629-40). The median PFS of 9.1 months with nazartinib was also similar to the median PFS of 10.1 months reported in the osimertinib arm of AURA3 (Mok et al. N Engl J Med 2017; 376: 629-40).

Similar responses were observed in patients regardless of whether their last prior systemic therapy was an EGFR TKI, suggesting that the observed efficacy is unlikely to be attributed to a “re-treatment effect”. Clinical activity in patients with EGFR T790M⁺ ex19del mutations appeared to be higher than in patients with EGFR T790M⁺ L858R mutations for both ORR and PFS, though the latter did not meet statistical significance. Differential activity of EGFR TKIs between ex19del and L858R mutations has been previously reported, albeit inconsistently (Zhang et al. PLoS One 2014;9:e107161; Yu et al. Chin J Cancer 2016; 35: 30,016-0086-2; Lin et al. Eur Respir J 2016; 48: PA4837). Other co-existing alterations detected had little bearing on quality of response to nazartinib.

Efficacy has been reported for osimertinib in patients with CNS metastases in AURA3, with the hazard ratio for PFS being comparable with that of the overall population (0.32 [95% CI, 0.21-0.49] vs. 0.30 [95% CI, 0.23-0.41] respectively) (Mok et al. N Engl J Med 2017; 376: 629-40). Lack of CNS activity with rociletinib may have contributed to the reduced confirmed response rates reported in mature data from the Phase I/II study. Rociletinib initially demonstrated an ORR of 59% in patients with EGFR T790M-mutant NSCLC progressing on prior EGFR TKI (Sequist et al. N Engl J Med 2015; 372: 1700-9); however, later analysis of mature data showed reduced confirmed response rates of 28-45% (Sequist et al. N Engl J Med 2016; 374: 2296-7; Business Wire. Clovis Oncology announces regulatory update for rociletinib NDA filing. Nov. 16, 2015: world wide web.

businesswire.com/news/home/20151116005513/en/). The authors hypothesized that early brain metastases may underlie the failure to confirm initial responses (Sequist et al. N Engl J Med 2016; 374: 2296-7). Following this, Clovis Oncology discontinued the clinical development of rociletinib (OncLive. Clovis Ends Development of Rociletinib in Lung Cancer. May 6, 2016: world wide web

://global.onclive.com/web-exclusives/clovis-ends-development-of-rociletinib-in-lung-cancer). By contrast, no patients with EGFR T790M⁺ NSCLC treated with nazartinib in this study experienced brain-only progression, and 14% of patients with baseline brain lesions had resolution of these lesions while on treatment with nazartinib. These data suggest that nazartinib exerts clinically-meaningful antitumor activity on lesions within the brain.

Nazartinib was generally well tolerated; most drug-related adverse events were low or moderate grade. Six patients experienced DLTs at doses 150 mg QD and above. The most common adverse events were rash, diarrhea, and pruritus; similar to those observed with other third-generation EGFR TKIs, such as osimertinib and olmutinib (Mok et al. N Engl J Med 2017; 376: 629-40; Park et al. J Thorac Oncol 2016; 11(4 Suppl): S113,0864(16)30243-X. Epub 2016 Apr. 15). Rash was the most common adverse event suspected to be drug-related. Rash occurring upon treatment with nazartinib is distinct from acneiform/pustular rash, which is often associated with treatment using EGFR TKIs that target EGFRwt. Rash upon nazartinib treatment was predominantly low-grade maculo-papular rash and usually acute and self-limiting, occurring during the first 4-6 weeks of treatment. This type of rash responded to systemic anti-allergic treatment and/or dose interruption or dose reduction and rarely recurred.

Two patients experienced HBV reactivation with nazartinib treatment, both of whom were treated at 225 mg QD, leading to hepatic failure and death in one patient. The precise mechanism of viral reactivation in these patients is not known, but may be related to the inhibitory effect of nazartinib on kinases within the Tec family such as Bruton's tyrosine kinase (BTK). Viral hepatitis reactivation has been reported in association with other anti-neoplastic therapies. Following the institution of HBV and HCV screening, monitoring, and management algorithms in this study, no additional cases of HBV reactivation were reported.

Furthermore, with the favorable safety profile observed, the efficacy achieved across all dose levels tested, and the selectivity of nazartinib for mutant EGFR over wild-type also indicate that a sufficient therapeutic window may exist to allow combination of nazartinib with other anti-neoplastic agents with acceptable tolerability. Such combination approaches will likely be necessary to overcome the TKI resistance such as MET amplification, a commonly reported mechanism of resistance to first- and third-generation EGFR TKIs (Sequist et al. Sci Transl Med 2011; 3: 75ra26; Piotrowska et al. J Clin Oncol 2017; 35: (suppl; abstr 9020); Oxnard et al. AACR Annual Meeting 2017; (abstr. 4112/22)). A separate study of nazartinib in combination with the MET inhibitor capmatinib (Phase I/II; NCT02335944) is ongoing in patients with EGFR-mutant NSCLC. Additional combinations may also be explored clinically, with a goal of preventing or delaying the emergence of treatment resistance in patients with advanced EGFR-mutant NSCLC.

Clinical Activity in Patients with Brain Metastases at Baseline

Patients with asymptomatic and/or previously-treated stable brain metastases were permitted in this study. Of 162 patients with EGFR T790M-mutant NSCLC who were third-generation EGFR TKI naïve, 46 (28%) had brain metastases at baseline. In 45 (98%) of these patients, brain lesions were recorded as non-target lesions only. In seven of these 45 patients (16%), brain non-target lesions became radiologically undetectable during treatment (1/9, 3/23, and 3/9 patients in the 100 mg, 150 mg, and 225 mg nazartinib groups, respectively). One such case was a 69-year-old Asian male patient with EGFR ex19del/T790M NSCLC who was previously treated with gefitinib and afatinib. He had numerous small brain metastases at baseline and had not previously received local therapy to the brain. After nazartinib treatment, the patient achieved an overall partial response of 11.1 months' duration, and brain metastases were no longer radiologically detectable by the first post-baseline evaluation.

After a median duration of follow-up of 29·6 months (all patients) and 29·2 months (patients with brain metastases at baseline), 118/162 patients (73%) overall and 33/46 (72%) patients with brain metastases at baseline had experienced disease progression. Of the 118 patients who progressed, only 19 (16%) experienced progression in the brain, and only 12 (10%) experienced progression in the brain without documented concurrent progression outside of the brain (five of these patients had brain metastases at baseline).

Tables: Summary of Brain Metastasis-Full Analysis Set

| | 75 mg EGF816 | | 100 mg EGF816 | | 150 mg EGF816 | |
|---|---|---|---|---|---|---|
| | QD | | QD | | QD | |
| | N = 12 | | N = 34 | | N = 68 | |
| | n = (%) | | n = (%) | | n = (%) | |
| Number of patients | 1 | (8.3) | 10 | (29.4) | 23 | (33.8) |
| with brain | | | | | | |
| metastasis at baseline | | | | | | |
| Number of patients with | 1 | (100.0) | 9 | (90.0) | 23 | (100.0) |
| brain metastasis in non- | | | | | | |
| target lesions only at | | | | | | |
| baseline | | | | | | |
| Number of patients with | 0 | (0.0) | 1 | (10.0) | 0 | (0.0) |
| brain metastasis in target | | | | | | |
| lesions only at baseline | | | | | | |
| Number of patients with | 0 | (0.0) | 0 | (0.0) | 0 | (0.0) |
| brain metastasis in both | | | | | | |
| lesions non-target and target | | | | | | |
| at baseline | | | | | | |
| Number of patients with | 0 | (0.0) | 1 | (10.0) | 1 | (4.3) |
| brain metastasis at | | | | | | |
| baseline who developed | | | | | | |
| new brain metastasis | | | | | | |
| Best response of brain | | | | | | |
| non-target lesions# | | | | | | |
| Absent/normalized | 0 | (0.0) | 1 | (11.1) | 3 | (13.0) |
| Present | 1 | (100.0) | 8 | (88.9) | 16 | (69.6) |
| Worsening | 0 | (0.0) | 0 | (0.0) | 3 | (13.0) |
| Unknown | 0 | (0.0) | 0 | (0.0) | 1 | (4.3) |
| Missing | 0 | (0.0) | 0 | (0.0) | 0 | (0.0) |
| Best % change from | | | | | | |
| baseline in brain | | | | | | |
| target lesions$ | | | | | | |
| >=−30 | 0 | (0.0) | 0 | (0.0) | 0 | (0.0) |
| −30 to <20 | 0 | (0.0) | 0 | (0.0) | 0 | (0.0) |
| >=20 | 0 | (0.0) | 1 | (100.0) | 0 | (0.0) |
| Number of patients without | 11 | (91.7) | 24 | (70.6) | 45 | (66.2) |
| brain metastasis at baseline | | | | | | |
| Number of patients without | 2 | (18.2) | 2 | (8.3) | 5 | (11.1) |
| brain metastasis at baseline | | | | | | |
| who developed new brain | | | | | | |
| metastasis | | | | | | |
| | 200 mg EGF816 | | 225 mg EGF816 | | 300 mg EGF816 | |
| | QD | | QD | | QD | |
| | N = 8 | | N = 24 | | N = 5 | |
| | n = (%) | | n = (%) | | n = (%) | |
| Number of patients with | 1 | (12.5) | 9 | (37.5) | 1 | (20.0) |
| brain metastasis at baseline | | | | | | |
| Number of patients with | 1 | (100.0) | 9 | (100.0) | 1 | (100.0) |
| brain metastasis in non- | | | | | | |
| target lesions only at | | | | | | |
| baseline | | | | | | |
| Number of patients with | 0 | (0.0) | 0 | (0.0) | 0 | (0.0) |
| brain metastasis in target | | | | | | |
| lesions only at baseline | | | | | | |
| Number of patients with | 0 | (0.0) | 0 | (0.0) | 0 | (0.0) |
| brain metastasis in both | | | | | | |
| non-target and target | | | | | | |
| lesions at baseline | | | | | | |
| Number of patients with | 0 | (0.0) | 0 | (0.0) | 0 | (0.0) |
| brain metastasis at baseline | | | | | | |
| who developed new brain | | | | | | |
| metastasis | | | | | | |
| Best response of brain | | | | | | |
| non-target lesions# | | | | | | |
| Absent/normalized | 0 | (0.0) | 3 | (33.3) | 0 | (0.0) |
| Present | 1 | (100.0) | 6 | (66.7) | 1 | (100.0) |
| Worsening | 0 | (0.0) | 0 | (0.0) | 0 | (0.0) |
| Unknown | 0 | (0.0) | 0 | (0.0) | 0 | (0.0) |
| Missing | 0 | (0.0) | 0 | (0.0) | 0 | (0.0) |
| Best % change | | | | | | |
| from baseline in | | | | | | |
| brain target lesions$ | | | | | | |
| >=−30 | 0 | (0.0) | 0 | (0.0) | 0 | (0.0) |
| −30 to <20 | 0 | (0.0) | 0 | (0.0) | 0 | (0.0) |
| >=20 | 0 | (0.0) | 0 | (0.0) | 0 | (0.0) |
| Number of patients without | 7 | (87.5) | 15 | (62.5) | 4 | (80.0) |
| brain metastasis at baseline | | | | | | |
| Number of patients without | 0 | (0.0) | 0 | (0.0) | 0 | (0.0) |
| brain metastasis at baseline | | | | | | |
| who developed | | | | | | |
| new brain metastasis | | | | | | |

| | 350 mg | | All | |
|---|---|---|---|---|
| | EGF816 QD | | patients | |
| | N = 11 | | N = 162 | |
| | n = (%) | | n = (%) | |
| Number of patients with brain | 1 | (9.1) | 46 | (28.4) |
| metastasis at baseline | | | | |
| Number of patients with brain | 1 | (100.0) | 45 | (97.8) |
| metastasis in non-target | | | | |
| lesions only at baseline | | | | |
| Number of patients with brain | 0 | (0.0) | 1 | (2.2) |
| metastasis in target lesions only | | | | |
| at baseline | | | | |
| Number of patients with brain | 0 | (0.0) | 0 | (0.0) |
| metastasis in both non-target | | | | |
| and target lesions at baseline | | | | |
| Number of patients with brain | 0 | (0.0) | 2 | (4.3) |
| metastasis at baseline who | | | | |
| developed new brain metastasis | | | | |
| Best response of brain | | | | |
| non-target lesions# | | | | |
| Absent/normalized | 0 | (0.0) | 7 | (15.6) |
| Present | 1 | (100.0) | 34 | (75.6) |
| Worsening | 0 | (0.0) | 3 | (6.7) |
| Unknown | 0 | (0.0) | 1 | (2.2) |
| Missing | 0 | (0.0) | 0 | (0.0) |
| Best % change from baseline | | | | |
| in brain target lesions$ | | | | |
| >= −30 | 0 | (0.0) | 0 | (0.0) |
| −30 to <20 | 0 | (0.0) | 0 | (0.0) |
| >= 20 | 0 | (0.0) | 1 | (100.0) |
| Number of patients without | 10 | (90.9) | 116 | (71.6) |
| brain metastasis at baseline | | | | |
| Number of patients without brain | 0 | (0.0) | 9 | (7.8) |
| metastasis at baseline who developed | | | | |
| new brain metastasis | | | | |

N: The total number of patients in the treatment group. It is the denominator for percentage (%) calculation.
n: Number of patients who are at the corresponding category.
#Use number of patients with brain non-target lesions at baseline as denominator.
$Use number of patients with brain target lesions at baseline as denominator.
EGFR T790M—patients or patients received prior third generation TKI are excluded.

Summary of Brain Metastasis Status at Disease Progression

| | 75 mg | | 100 mg | | 150 mg | | 200 mg | | 225 mg | |
|---|---|---|---|---|---|---|---|---|---|---|
| | EGF816 | | EGF816 | | EGF816 | | EGF816 | | EGF816 | |
| | QD | | QD | | QD | | QD | | QD | |
| | N = 12 | | N = 34 | | N = 68 | | N = 8 | | N = 24 | |
| | n = (%) | | n = (%) | | n = (%) | | n = (%) | | n = (%) | |
| All patients | | | | | | | | | | |
| Progressed | 7 | (58.3) | 22 | (64.7) | 55 | (80.9) | 7 | (87.5) | 17 | (70.8) |
| Not progressed | 5 | (41.7) | 12 | (35.3) | 13 | (19.1) | 1 | (12.5) | 7 | (29.2) |
| Among patients who progressed | | | | | | | | | | |
| A: New brain metastatsis occurred at first | 2 | (28.6) | 5 | (22.7) | 9 | (16.4) | 1 | (14.3) | 1 | (5.9) |
| PD or non-target lesion brain metastasis | | | | | | | | | | |
| worsened at first PD or sum of brain target | | | | | | | | | | |
| lesion diameters increases by at | | | | | | | | | | |
| least 20% at first PD | | | | | | | | | | |
| B: Sum of target lesion diameters outside | 6 | (85.7) | 19 | (86.4) | 49 | (89.1) | 6 | (85.7) | 16 | (94.1) |
| of brain increased by at least 20% at first | | | | | | | | | | |
| PD or worsening non-target lesion response | | | | | | | | | | |
| non-brain outside of brain at first PD or | | | | | | | | | | |
| new lesion at first PD | | | | | | | | | | |
| Only A, not B | 1 | (14.3) | 3 | (13.6) | 5 | (9.1) | 1 | (14.3) | 1 | (5.9) |
| Only B, not A | 5 | (71.4) | 17 | (77.3) | 45 | (81.8) | 6 | (85.7) | 16 | (94.1) |
| Both A and B | 1 | (14.3) | 2 | (9.1) | 4 | (7.3) | 0 | (0.0) | 0 | (0.0) |
| Among patients with baseline | | | | | | | | | | |
| brain metastasis | | | | | | | | | | |
| Progressed | 1 | (100.0) | 7 | (70.0) | 16 | (69.6) | 1 | (100.0) | 7 | (77.8) |
| Not progressed | 0 | (0.0) | 3 | (30.0) | 7 | (30.4) | 0 | (0.0) | 2 | (22.2) |
| Among patients with baseline brain | | | | | | | | | | |
| metastasis who progressed | | | | | | | | | | |
| A: New brain metastatsis occurred at first | 0 | (0.0) | 3 | (42.9) | 4 | (25.0) | 1 | (100.0) | 1 | (14.3) |
| PD or non-target lesion brain metastasis | | | | | | | | | | |
| worsened at first PD or sum of brain | | | | | | | | | | |
| target lesion diameters increases by | | | | | | | | | | |
| at least 20% at first PD | | | | | | | | | | |
| B: Sum of target lesion diameters outside | 1 | (100.0) | 6 | (85.7) | 15 | (93.8) | 0 | (0.0) | 6 | (85.7) |
| of brain increases by at least 20% at | | | | | | | | | | |
| first PD or worsening non-target lesion | | | | | | | | | | |
| response outside of brain at first PD or | | | | | | | | | | |
| new non-brain lesion at first PD | | | | | | | | | | |
| Only A, not B | 0 | (0.0) | 1 | (14.3) | 1 | (6.3) | 1 | (100.0) | 1 | (14.3) |
| Only B, not A | 1 | (100.0) | 4 | (57.1) | 12 | (75.0) | 0 | (0.0) | 6 | (85.7) |
| Both A and B | 0 | (0.0) | 2 | (28.6) | 3 | (18.8) | 0 | (0.0) | 0 | (0.0) |

| | 300 mg | | 350 mg | | | |
|---|---|---|---|---|---|---|
| | EGF816 QD | | EGF816 QD | | All patients | |
| | N = 5 | | N = 11 | | N = 162 | |
| | n = (%) | | n = (%) | | n = (%) | |
| All patients | | | | | | |
| Progressed | 4 | (80.0) | 6 | (54.5) | 118 | (72.8) |
| Not progressed | 1 | (20.0) | 5 | (45.5) | 44 | (27.2) |
| Among patients who progressed | | | | | | |
| A: New brain metastatsis occurred at first PD or non- | 1 | (25.0) | 0 | (0.0) | 19 | (16.1) |
| target lesion brain metastasis worsened at first PD or sum | | | | | | |
| of brain target lesion diameters increases by at least 20% | | | | | | |
| at first PD | | | | | | |
| B: Sum of target lesion diameters outside of brain | 3 | (75.0) | 6 | (100.0) | 105 | (89.0) |
| increased by at least 20% at first PD or worsening non- | | | | | | |
| target lesion response outside of brain at first PD or new | | | | | | |
| non-brain lesion at first PD | | | | | | |
| Only A, not B | 1 | (25.0) | 0 | (0.0) | 12 | (10.2) |
| Only B, not A | 3 | (75.0) | 6 | (100.0) | 98 | (83.1) |
| Both A and B | 0 | (0.0) | 0 | ( 0.0) | 7 | (5.9) |
| Among patients with baseline brain metastasis | | | | | | |
| Progressed | 1 | (100.0) | 0 | (0.0) | 33 | (71.7) |
| Not progressed | 0 | (0.0) | 1 | (100.0) | 13 | (28.3) |
| Among patients with baseline | | | | | | |
| brain metastasis who progressed | | | | | | |
| A: New brain metastatsis occurred at first PD or non- | 1 | (100.0) | 0 | (0.0) | 10 | (30.3) |
| target lesion brain metastasis worsened at first PD or sum | | | | | | |
| of brain target lesion diameters increases by at least 20% | | | | | | |
| at first PD | | | | | | |
| B: Sum of target lesion diameters outside of brain | 0 | (0.0) | 0 | (0.0) | 28 | (84.8) |
| increases by at least 20% at first PD or worsening non- | | | | | | |
| target lesion response outside of brain at first PD or new | | | | | | |
| non-brain lesion at first PD | | | | | | |
| Only A, not B | 1 | (100.0) | 0 | (0.0) | 5 | (15.2) |
| Only B, not A | 0 | (0.0) | 0 | (0.0) | 23 | (69.7) |
| Both A and B | 0 | (0.0) | 0 | (0.0) | 5 | (15.2) |

N: The total number of patients in the treatment group. It is the denominator for percentage (%) calculation.
n: Number of patients who are at the corresponding category.
EGFR T790M—patients or patients received prior third generation TKI are excluded.

In conclusion, these results demonstrate promising clinical activity of nazartinib in patients with EGFR-mutant NSCLC patients. In particular, the results indicate that nazartinib has the potential to be effective in the treatment or prevention of CNS metastasis, brain metastasis and leptomeningeal metastasis, particularly in patients suffering from EGFR-mutant NSCLC.

### Example 2

### Phase II Trial of Single Agent Nazartinib in Adult Patients with EGFR-Mutated Non-Small Lung Cancer (NSCLC) who had not Received Prior Treatment

This phase I/II multicentre study of nazartinib was conducted in treatment-naive patients with advanced EGFR-mutant NSCLC harbouring activating EGFR L858R and/or ex19del mutations.

All 45 patients received the recommended phase II oral dose of 150 mg once daily on a continuous schedule. Anti-tumour activity, including overall response rate (ORR) per RECIST v1.1, as assessed by blinded independent central review (BICR), served as the primary objective, and secondary objectives included safety, tolerability, and pharmacokinetics. The median age was 64 years, 60% of patients in the trial were female, and 62% were Asian. Fifty-eight percent had ECOG performance status 1 and 18 (45%) patients had brain metastasis at baseline. EGFR mutations were ex19del in 56% of patients, L858R in 40%, and 4% of patients had other EGFR mutations. Twenty-nine of 45 patients demonstrated a response to nazartinib, yielding an ORR of 64% (95% confidence interval [CI], 49%-78%). One patient achieved complete response.

At data cutoff on 22 Mar. 2018, responses were ongoing in 27 of the 29 responding patients. The 6-month duration of response rate (DoR) was 91%, and the median DoR was not estimable (NE) and not available yet. The disease control rate was 93%.

The 6-month progression-free survival rate was 83% (median NE) and the 6-month overall survival rate was 95% (median NE) with nazartinib.

Evaluation of the 17 patients with baseline brain metastasis in non-target lesions showed 9 (53%) patients had resolution of brain metastasis. One of the 27 patients without baseline brain metastasis developed a new brain metastasis on study. At data cutoff on 22 Mar. 2018, responses were ongoing in 27 of the 29 responding patients. The 6-month duration of response rate (DoR) was 91%, and the median DoR was not estimable (NE). The disease control rate was 93%.

The 6-month progression-free survival rate was 83% (median NE) and the 6-month overall survival rate was 95% (median NE) with nazartinib.

The data point to an effective third generation EGFR-TKI with good brain penetration. Nazartinib demonstrated promising efficacy, and provided durable responses in treatment-naive patients with advanced EGFR-mutant NSCLC, including patients with baseline brain metastases.

Preliminary Data on Patients Enrolled 15 Weeks before Cut-Off Date

The following data set, based on evaluable patients enrolled 15 weeks before cut-off date further indicates that nazartinib may be particularly useful in the treatment or prevention of a metastasis which is Central Nervous System (CNS) metastasis, brain metastasis and leptomeningeal metastasis.

Brain metastases at baseline were assessed by computed tomography (CT)/magnetic resonance imaging (MRI). Ten out of 24 (42%) evaluable patients had brain metastases at baseline.

Nazartinib was effective both in patients with and without brain metastasis at baseline (see Table below). Complete and/or partial responses were achieved in 5/10 (ORR 50%) and 11/14 (ORR 79%) patients with and without brain metastases at baseline by BIRC, respectively. The DCR (stable disease, partial response and complete response) was similar in patients with (90%) and without (100%) brain metastases by BIRC. This suggests that nazartinib is crossing into the brain and effective in treating brain tumors.

TABLE
Best Overall Response by Brain Metastases at
Baseline per BIRC in accordance with RECIST v1.1

| | BIRC (FAS) | | | |
|---|---|---|---|---|
| | n = 24 | | | |
| | Brain | | Brain | |
| | metastases | | metastases | |
| | present | | absent | |
| Evaluable patients, n (%)* | 10 | (41.7) | 14 | (58.3) |
| Best overall response, n (%) | | | | |
| Complete response (CR) | 0 | | 1 | (7.1) |
| Partial response (PR) | 5 | (50.0) | 10 | (71.4) |
| Stable Disease (SD) | 4 | (40.0) | 2 | (14.3) |
| Progressive disease (PD) | 1 | (10.0) | 0 | |
| Non-CR/non-PD (NCRNPD) | 0 | | 1 | (7.1) |
| Disease control rate, n (%)^{†} | 9 | (90.0) | 14 | (100) |
| [95% CI] | [55.5-99.7] | | [76.8-100] | |
| Overall response rate, n (%) | 5 | (50.0) | 11 | (78.6) |

| | | |
|---|---|---|
| [95% CI] | [18.7-81.3] | [49.2-95.3] |

*Evaluable patients: patients enrolled 15 weeks before cutoff date. This total is used for percentage calculation.
^{†}CR + PR + SD + NCRNPD.
BIRC, Blinded Independent Review Committee; CT, computed tomography; FAS, full analysis set;
MRI, magnetic resonance imaging; RECIST, Response Evaluation Criteria in Solid Tumors.
Brain metastases at baseline were assessed by CT/MRI.

In addition, a 49 year old female patient with EGFR-mutated (ex19del, G719S/A/C) lung adenocarcinoma, AJCC stage IV (brain, bone, liver) showed a PR as best overall response. Brain post-baseline scan performed at Cycle 5 Day 1 (study day 113) showed complete normalization of brain lesion, compared to baseline by BIRC. The patient had had no prior treatment for brain metastases, thus suggesting that this result was attributable to nazartinib and not a delayed response to radiological treatment or other treatment.

### Example 3

### A Randomized, Open-label, Phase III Study of Single Agent Nazartinib Versus Investigator's Choice (Erlotinib or Gefitinib) as First-Line Treatment in Patients with Locally Advanced or Metastatic Non-Small Cell Lung Cancer Harboring EGFR Activating Mutations

The purpose of this study is to evaluate the superiority of single agent EGF816 assessed by PFS as determined by central BIRC, compared with investigator's choice (erlotinib or gefitinib) in patients with locally advanced or metastatic NSCLC who are treatment naïve and whose tumors harbor EGFR activating mutations (L858R or ex19del).

The primary objective of this study is to compare the efficacy of single agent EGF816 compared to investigator's choice (erlotinib or gefitinib) as measured by PFS as per central BIRC and according to Response Evaluation Criteria in Solid Tumors (RECIST 1.1). The key secondary objective of this study is to compare Overall Survival (OS) of single agent EGF816 compared to investigator's choice (erlotinib or gefitinib). The other secondary objectives are as follows:
To further assess the efficacy of single agent EGF816 compared to investigator's choice (erlotinib or gefitinib) by PFS as determined by investigators, ORR, DoR, Disease Control Rate (DCR) and time to response (TTR) as determined by central BIRC.
To assess the long-term benefit of EGF816
To assess the antitumor activity of EGF816 in the central nervous system (CNS) compared to investigator's choice (erlotinib or gefitinib), as measured by time to progression in the CNS, ORR in the CNS, and DoR in the CNS as determined by central neuro-radiologist BIRC according to modified RECIST 1.1 for patients with CNS disease at baseline.
To characterize the pharmacokinetics (PK) of single agent EGF816.
To assess the impact of single agent EGF816 compared investigator's choice (erlotinib or gefitinib) on patient reported outcomes (PRO), including patients' disease-related symptoms and Health Related Quality of Life (HRQoL).
To assess the safety and tolerability profile of single agent EGF816 compared to investigator's choice (erlotinib or gefitinib).

The study population includes adult patients with locally advanced or metastatic NSCLC with EGFR activating mutations (L858R or ex19del) and who are treatment-naïve.

Patients are randomized in a 1:1 ratio to either: Arm A (investigator's choice): erlotinib 150 mg QD given without food or gefitinib 250 mg QD with or without food or Arm B: EGF816 50 mg QD, or 75 mg QD, or 100 mg QD, or 150 mg QD with or without food.

Background

Osimertinib (Tagrisso®), one of the third-generation EGFR inhibitors, is indicated for metastatic EGFR T790M mutation-positive NSCLC, in patients who have progressed during or after EGFR-TKI therapy (Jänne et al. 2015, N Engl J Med., vol. 372 (18), pp. 1689-1699).

Osimertinib reported positive results in first-line treatment of EGFR mutant NSCLC in the FLAURA (NCT02296125) study: investigator reported median progression-free survival was significantly longer with osimertinib than with standard EGFR-TKIs (18.9 months vs. 10.2 months; HR for disease progression or death, 0.46; 95% confidence interval [CI], 0.37 to 0.57; P<0.001). The objective response rate (ORR) was similar in the two groups: 80% with osimertinib and 76% with standard EGFR-TKIs (odds ratio, 1.27; 95% CI, 0.85 to 1.90; P=0.24). The median duration of response was 17.2 months (95% CI, 13.8 to 22.0) with osimertinib versus 8.5 months (95% CI, 7.3 to 9.8) with standard EGFR-TKIs. Data on overall survival were immature at the interim analysis (25% maturity). The survival rate at 18 months was 83% (95% CI, 78 to 87) with osimertinib and 71% (95% CI, 65 to 76) with standard EGFR-TKIs (hazard ratio for death, 0.63; 95% CI, 0.45 to 0.88; P=0.007 [non-significant in the interim analysis]). The most commonly reported adverse events (AEs) due to any cause were rash or acne (58% in the osimertinib group and 78% in the standard EGFR-TKI group), diarrhea (58% and 57%, respectively), and dry skin (36% in each group). AEs of grade 3 or higher were less frequent with osimertinib than with standard EGFR-TKIs (34% vs. 45%) (Soria et al. 2018, N Engl J Med, vol. 378 (2), pp. 113-125).

Erlotinib (Tarceva®) is an orally active, potent, selective inhibitor of the EGFR TK. Erlotinib has been approved in most countries for the first-line treatment of patients with metastatic NSCLC whose tumors exhibited EGFR ex19del or L858R substitution mutations. For additional information, refer to erlotinib (Tarceva®) local label.

The safety and efficacy of erlotinib as monotherapy for the first-line treatment of patients with metastatic NSCLC containing EGFR ex19del or L858R substitution mutations was demonstrated in a randomized, open-label, clinical trial conducted in Europe (EURTAC study) where 174 patients were randomized 1:1 to receive erlotinib 150 mg once daily or platinum-based doublet chemotherapy (n=88). The median PFS was 9.7 months for erlotinib compared to 5.2 months for platinum-based chemotherapy as assessed by the investigator, with no improvement in OS (Rosell et al. 2012, Lancet Oncol. vol. 13 (3), pp. 239-246).

The most common adverse reactions (≥20%) with erlotinib from a pooled analysis were rash (acneiform in most cases), diarrhea, anorexia, fatigue, dyspnea, cough, nausea, and vomiting. ILD occurred in 1.1% of patients.

Gefitinib (Iressa®) is a potent and selective reversible inhibitor of the EGFR TK. Gefitinib has been approved in most countries for the first-line treatment of patients with NSCLC whose tumors have EGFR ex19del or L858R substitution mutations.

The randomized phase III first-line IPASS study was conducted in patients in Asia with advanced (stage IIIB or IV) NSCLC of adenocarcinoma histology who were ex-light smokers. Patients were randomized 1,217 (1:1) to received gefitinib or carboplatin/paclitaxel. In patients with EGFR activating mutations, gefitinib demonstrated superior PFS with 9.5 months versus 6.3 months, ORR (71.2% versus 47.3%), with no significant difference in overall survival compared to carboplatin/paclitaxel.

The most frequently reported adverse drug reactions (occurring in more than 20% of the patients) among 2463 patients treated with gefitinib, were diarrhea and skin reactions (including rash (acneiform in most cases), acne, dry skin and pruritus). Adverse drug reactions usually occur within the first month of therapy and are generally reversible. Approximately 8% of patients had a severe adverse drug reaction (grade 3 or 4). Approximately 3% of patients stopped therapy due to an adverse drug reaction. ILD occurred in 1.3% of patients, often severe (CTC grade 3-4). Cases with fatal outcomes have been reported.

Rationale for the Study Design

This is a global, open-label, multicenter, active-controlled, 2-arm, phase III randomized study designed to compare the efficacy and safety of single agent EGF816 versus investigator's choice (erlotinib or gefitinib) in treatment-naïve patients with locally advanced or metastatic NSCLC, stage IIIB/IIIC (not amenable for definitive multi-modality therapy), or stage IV (Detterbeck et al. 2017, Chest, vol. 151 (1), pp. 193-203) with EGFR activating mutation (L858R or ex19del).

The primary objective of the study is to assess the efficacy of single agent EGF816 compared to the investigator's choice (erlotinib or gefitinib) as measured by PFS as per central BIRC and according to Response Evaluation Criteria in Solid Tumors (RECIST 1.1).

Patients are randomized in 1:1 fashion to one of two treatment arms:
Arm A: Reference arm (investigator's choice) erlotinib at 150 mg QD or gefitinib at 250 mg QD;
Arm B: EGF816 at 50, 75, 100 or 150 mg QD.

Rationale for Dose and Regimen Selection

The overall benefit/risk assessment of EGF816 is considered favorable for the treatment of patients with treatment-naïve locally advanced or metastatic NSCLC tumors harboring EGFR activating mutations. Based on the results of the study CEGF816X2101, the EGF816 dose of 150 mg QD was selected as RP2D as it was well tolerated and demonstrated antitumor-activity, in 2^{nd} line T790M positive and 1^{st} line EGFR mutated NSCLC patients enrolled in the phase I (dose-escalation part) and II (dose-expansion part) of the study. Study discontinuation rates were low, with 7.5% (n=3) discontinuing in the phase II (dose-expansion part) (2.5% due to adverse event).

Additionally, exploratory dose exposure-response analysis were also conducted using efficacy data (confirmed response) and safety data (rash) from the phase I (dose-escalation part) of the study CEGF816X2101, confirming 150 mg QD as optimal dose for EGF816 monotherapy.

Overall, based on the efficacy and safety data from phase I (dose-escalation) and phase II (dose-expansion), and dose/exposure-response analysis, 150 mg QD was selected as the dose for this phase III study which should provide a favorable benefit/risk ratio to patients with treatment-naïve locally advanced or metastatic NSCLC harboring EGFR activating mutations.

In conclusion, nazartinib, at the dose of 150 mg QD was efficacious in patients with advanced EGFR mutant NSCLC, with a manageable safety profile both in pretreated and treatment-naive setting, and 150 mg QD is the dose used in this phase III study conducted in front line EGFR mutant NSCLC.

Methods

Description of Study Design

This is an open-label, multicenter, active-controlled, 2-arm, phase III randomized study to evaluate the efficacy and safety of EGF816 compared to investigator's choice (erlotinib or gefitinib) in treatment-naïve adult patients with locally advanced or metastatic NSCLC, stage IIIB/IIIC (not amenable to definitive multi-modality therapy including surgery) or stage IV, harboring EGFR-activating mutation (L858R or ex19del).

Patients are randomized in a 1:1 ratio to either:
Arm A (investigator's choice): erlotinib 150 mg QD given without food or gefitinib 250 mg QD with or without food or to
Arm B: EGF816 50, 75, 100 or 150 mg QD with or without food.

Cross-over from one treatment arm to the other is allowed; within Arm A (investigator's choice), it is not permitted to switch to the other drug (either erlotinib or gefitinib) during the study.

The study includes the following periods:
Molecular pre-screening period
Screening period (following signature of main Informed Consent Form (ICF) up to 28 days prior to randomization)
Treatment period until progressive disease, unacceptable toxicity or discontinuation from the study treatment for any other reason
Safety follow-up period (30 days after the last dose of study medication)
Post-treatment follow-up for patients who discontinue the study treatment for reasons other than withdrawal of consent, progressive disease, pregnancy, start of new anti-neoplastic therapy, lost to follow-up, study terminated by the sponsor or death
Survival follow-up, including PFS2.

Inclusion Criteria

Patients eligible for inclusion in this study have to meet all of the following criteria:
Written informed consent obtained prior to any screening procedures.
Histologically documented locally advanced or metastatic, stage IIIB/IIIC or stage IV NSCLC with documented EGFR activating mutation (L858R or ex19del)
Provision of a tumor tissue sample to allow for retrospective analysis of EGFR mutation status
No prior treatment with any systemic antineoplastic therapy in the advanced setting
Recovered from all toxicities related to prior treatment
Presence of at least one measurable lesion according to RECIST 1.1
Eastern Cooperative Oncology Group (ECOG) performance ≤1
Meet the following laboratory values at the screening visit:
   Absolute Neutrophil Count≥1.5×109/L
   Platelets≥75×109/L
   Hemoglobin (Hgb)≥9 g/dL
   Creatinine Clearance≥45 mL/min using Cockcroft-Gault formula
   Total bilirubin≤1.5×ULN
   Aspartate transaminase (AST)≤3.0×ULN, except for patients with liver metastasis, who may only be included if AST≤5.0×ULN
   Alanine transaminase (ALT)≤3.0×ULN, except for patients with liver metastasis, who may only be included if ALT≤5.0×ULN

Exclusion Criteria
Prior treatment with EGFR-TKI.
Known T790M positive mutation. Any other known EGFR activating mutations other than L858R or ex19del. Patients whose tumors harbor other EGFR mutations concurrent with L858R or ex19del EGFR mutations are eligible.
Symptomatic brain metastases
History of interstitial lung disease or interstitial pneumonitis
Any medical condition that would, in the investigator's judgment, the patient's in the study due to safety concerns, compliance with clinical study procedures or interpretation of study results
Presence or history of a malignant disease other than NSCLC that has been diagnosed and/or required therapy within the past 3 years.
Presence of clinically significant ophthalmologic abnormalities
Bullous and exfoliative skin disorders of any grade
Presence or history of microangiopathic hemolytic anemia with thrombocytopenia.
Known history of testing positive for human immunodeficiency virus (HIV) infection
Cardiac or cardiac repolarization abnormality
Major surgery: ≤4 weeks to starting study treatment or who have not recovered from side effects of such procedure.
Unable or unwilling to swallow tablets or capsules
Female patients who are either pregnant or nursing
Women of child bearing potential who refuse or are not able to use a highly effective method of contraception as defined in the study protocol.
Sexually active males unless they use a condom during intercourse while taking drug and for 3 months after the last dose of study treatment.

Dosing Regimen

EGF816, erlotinib or gefitinib is administered orally once per day on a continuous dosing schedule. A complete cycle of treatment is defined as 21 days of daily dosing (Table 8).

TABLE 8
Dose and treatment schedule.

| Study | Pharmaceutical form and | | Frequency and/or |
|---|---|---|---|
| treatments | route of administration | Dose | Regimen |
| EGF816 | Capsule for oral use, | 150 mg | Daily |
| | with or without food | | (21 day cycles) |
| Gefitinib | Tablet for oral use, | 250 mg | Daily |
| | with or without food | | (21 day cycles) |
| Erlotinib | Tablet for oral use, | 150 mg | Daily |
| | without food | | (21 day cycles) |

TABLE 9
Other doses envisaged for
EGF816, erlotinib and gefitinib.

| | Starting | | |
|---|---|---|---|
| | dose | Dose | Dose |
| | level | level-1 | level-2 |
| EGF816 | 150 mg QD | 100 mg QD | 75 mg QD |
| Erlotinib | 150 mg QD | 100 mg QD | 50 mg QD |
| Gefitinib | 250 mg QD | NA | NA |

*Dose reduction should be based on the worst toxicity at the last dose.

1. A method of treating or preventing metastasis in a patient in need thereof, by administering a therapeutically effective amount of nazartinib, or a pharmaceutically acceptable salt thereof, wherein the metastasis is selected from central nervous system (CNS) metastasis, brain metastasis and leptomeningeal metastasis, and a metastasis resulting from a primary lesion selected from non-small lung cancer (NSCLC), NSCLC which harbors an exon 19 deletion, and NSCLC which harbors an exon 21 (L858R) substitution.

2. The method of claim 1, wherein nazartinib is in its mesylate salt form.

3. The method of claim 1, wherein the patient is a patient with locally advanced or metastatic non-small cell lung cancer (NSCLC).

4. The method of claim 1 wherein the NSCLC harbors an EGFR-activating mutation.

5. The method of claim 4, wherein the EGFR-activating mutation is an L858R mutation.

6. The method of claim 4, wherein the EGFR-activating mutation is an exon 19 deletion mutation.

7. The method of claim 1, wherein the patient is NSCLC patient who has progressed to develop brain metastasis, CNS metastasis and/or leptomeningeal metastasis.

8. The method of claim 1, wherein the Progression Free Survival (PFS) of the patient is improved in relation to the PFS obtained following treatment with erlotinib or gefitinib.

9. The method of claim 1, wherein the overall survival (OS) of the patient is improved in relation to the OS obtained following treatment with erlotinib or gefitinib.

10. The method of claim 1, wherein the overall response rate (ORR) of the patient is improved in relation to the ORR obtained following treatment with erlotinib or gefitinib.

11. The method of claim 1, wherein the time to progression (TPP) in CNS or brain is increased compared to the TPP in CNS or brain following treatment with erlotinib, gefitinib or osimertinib.

12. The method of claim 1, wherein the CNS or brain ORR is increased compared to the CNS ORR with erlotinib, gefitinib or osimertinib treatment.

13. The method of claim 1, wherein the CNS or brain duration of response (DoR) is increased compared to the CNS or brain DoR with erlotinib, gefitinib or osimertinib treatment.

14. The method of claim 1, wherein nazartinib is used as monotherapy.

15. A method of treating or preventing central nervous system (CNS) metastases, including brain metastases, by administering a therapeutically effective amount of Nazartinib, or a pharmaceutically acceptable salt thereof, in the first-line treatment of patients with metastatic non-small cell lung cancer (NSCLC) wherein the cancer harbors an EGFR mutation, an exon 19 deletion or an exon 21 (L858R) substitution.

16. The method of claim 15, wherein nazartinib is used as monotherapy for the treatment of NSCLC or as part of a combination therapy for the treatment of NSCLC.

17. The method of claim 1 wherein nazartinib is administered at a total dose which is selected from a range of about 50, 150 to about 200 mg, daily.

18. A method of treating a patient having NSCLC, comprising selectively administering a therapeutically effective amount of nazartinib, or a pharmaceutically acceptable salt thereof, to a patient having previously been determined to have an exon 19 deletion or exon 21 (L858R) substitution EGFR mutation.

19. A method of treating a patient having NSCLC, comprising:
(a) determining or having determined that the patient has an exon 19 deletion or exon 21 (L858R) substitution EGFR mutation; and
(b) administering a therapeutically effective amount of nazartinib, or a pharmaceutically acceptable salt thereof, to said patient.

20. A method of treating a patient having NSCLC, comprising selecting a patient for treatment based on the patient having been previously determined to have an exon 19 deletion or exon 21 (L858R) substitution EGFR mutation, and administering a therapeutically effective amount of nazartinib, or a pharmaceutically acceptable salt thereof, to said patient.

21. A method of claim 18 wherein the patient is a patient with a metastasis which is selected from central nervous system (CNS) metastasis, brain metastasis and leptomeningeal metastasis.

22. The method of claim 18 wherein the therapeutically effective amount is selected from a range of about 50, 150 to about 200 mg, administered once per day.

23. (canceled)
